(19) United States
(12) Patent Application Publication
(10) Pub. No.: US 2018/0292413 A1
(43) Pub. Date: Oct. 11, 2018

(54) Rigorous method and apparatuses for the analysis of complex mixtures of organic molecules with an enhanced degree of information extraction

(71) Applicant: Andreas Hieke, San Mateo, CA (US)

(72) Inventor: Andreas Hieke, San Mateo, CA (US)

(21) Appl. No.: 16/007,674

(22) Filed: Jun. 13, 2018

Related U.S. Application Data
(63) Continuation of application No. 15/456,206, filed on Mar. 10, 2017.
(60) Provisional application No. 62/307,470, filed on Mar. 12, 2016.

(51) Int. Cl. G01N 33/68; G01N 35/00; C12Q 1/6872; G16H 10/40; G16H 50/20; G06F 19/18

(52) U.S. Cl. CPC G01N 33/6848; G01N 35/0099; C12Q 1/6872; G01N 35/00871; G01N 2800/52; G01N 2570/00; G01N 2560/00; G01N 2440/00; G16H 50/20; G01N 2800/56; G01N 2800/60; G06F 19/18; G16H 10/40

(57) ABSTRACT

Disclosed are a rigorous method and apparatuses for the comprehensive analysis of complex mixtures of organic molecules, specifically biopolymers, in some embodiments predominantly mixtures of proteins, which in some embodiments ultimately serve to obtain the information constituting a biomarker, or similar biological signature, or to monitor health or ageing.

In some embodiments such signatures or patterns may indicate the presence, stage, or type of a disease, the expected or actual response to drugs. In some other embodiments such a method and apparatuses may serve to monitor and/or control cell development. In some other embodiments such a method and apparatuses may serve to develop and use means to measure, influence, or control the speed or degree of aging of cells or organisms.

In some embodiments such a method and apparatuses may serve to determine at least in part the nature of biological hazards, bioterrorism threats, or biological weapons, including those based on bacteria and viruses. In some embodiments such a method and apparatuses may serve to select, develop, and or optimize countermeasures to biological hazards, bioterrorism threats, or biological weapons, including those based on bacteria and viruses.

In other embodiments such a method and apparatuses may be used to assess the expected performance level of a human or animal for a specific task or for a class or problems.

### INCORPORATION BY REFERENCE TO ANY PRIORITY APPLICATIONS

Any and all applications for which a foreign or domestic priority claim is identified in the Application Data Sheet as filed with the present application are hereby incorporated by reference. In particular, this application is a continuation of U.S. patent application Ser. No. 15/456,206 filed Mar. 10, 2017, which claims the benefit of U.S. provisional patent application No. 62/307,470 filed Mar. 12, 2016, the disclosures of which is incorporated herein by reference.

### BACKGROUND OF THE INVENTION

### Field of the Invention

Much financial effort and attention has been given to achieving relatively inexpensive and rapid DNA sequencing, specifically also of human DNA, in many cases with hopes of ultimately making clinical use of such technology. The first attempt to obtain a human reference genome was an effort of more than a decade (1990 to 2003) and is estimated to have required about $3 billion in government funding. (A similar, although less costly and less comprehensive, privately funded effort was performed simultaneously.)

Due to massive investments, remarkable progress in instrumentation has been achieved during the last 15 years, which has resulted in an exponential decrease in costs and increase in speed thanks to fully automated high-throughput DNA sequencing machines. At the beginning of 2014 Illumina introduced the ‘HiSeq X Ten Sequencer’ which can, on average (if using 10 machines in parallel), sequence entire human genomes for less than $1,000 and in about half an hour.

While without any doubt inexpensive DNA sequencing will be of great practical importance for future medical and biological research, many of the original hopes to clinically use genetic information (by itself) for short and medium term health predictions have thus far only partially been fulfilled and are likely to remain illusive for principle reasons in many cases.

It is from a theoretical standpoint clear that the missing information is (thus far) hidden in protein abundance levels as well as in chemical modifications on proteins (posttranslational modifications). The challenge when analyzing protein mixtures obtained from nature is twofold: complexity and dynamic range. In addition, the changes in abundance and composition of proteins during the life of an organism are far more dynamic compared to changes of DNA. Due to the enormous complexity of human proteins (several millions) and their huge dynamic range (at least 10 orders of magnitude in human blood) protein mixture analysis represents an extraordinarily complicated metrology problem.

After the introduction of high throughput DNA sequencing, the next technologically feasible long term goal, likely achievable within less than a decade, must now be a comprehensive “$1000 human protein profile” based on orders of magnitude faster and intrinsically quantitative (label free) instruments to analyze complex protein mixtures.

In some embodiments the disclosed invention provides elements to at least approach or even realize this and other goals.

To illustrate the transformative impact of groundbreaking inventions a few historic examples shall be given henceforth. The history of science and technology is filled with examples where a substantial increase in performance of a measuring instrument, or a machine (incl. weapons) subsequently enabled fundamental discoveries, caused the transformation of entire industries, opened up entirely new commercial fields, or helped win wars.

One of the contributing reasons why Great Britain became a dominating world power in the 18th century was that ships of the British Navy had superior navigation capabilities compared to ships from other navies. They possessed marine chronometers, built by the carpenter John Harrison, which incorporated various motion and temperature compensation methods and which could therefore measure time with orders of magnitude higher accuracy than conventional clocks.

The collaboration between Ernst Abbe (providing an improved theory behind optical image formation, as well as novel lens designs such as apochromats), Carl Zeiss, and later Otto Schott (developing borosilicate crown glass with low optical dispersion), ultimately resulted in the design and production of reliable oil immersion microscopes, which operated close to the theoretical optical resolution limit. These instruments enabled numerous discoveries related to microbiology and infectious diseases, including Robert Koch's discovery of the tuberculosis bacterium in 1882.

Frits Zernike's invention the (optical) phase contrast microscope (Zernike, 1934, Nobel Lecture 1953) enabled the optical visualization of the internal structure of cells with largely improved contrast while avoiding usually lethal staining, which ushered in the area of research on living cells under microscopic observation.

During the early phases of the development of electron microscopes (incl. TEM, SEM, and STEM) during the 1930's and early 1940's (with major contributions by Hiller, Knoll, Ruska, Scherzer, Prebus, v. Borris, v. Ardenne, and others) serious skepticism existed if—although theoretically expected—images with significantly higher than optical resolution could practically be obtained. In Ernst Ruska's own words this was in fact considered “a pipe dream” by most experts at the time (Ruska, Nobel lecture 1986). Nevertheless, this lofty goal was of great interest, since some of the driving motivations behind these efforts, besides emanating from material sciences, came from biology, namely the desire to image viruses and bacteria. Moreover, there were fundamental doubts if biological samples could ever be imaged with electron beams and in vacuum. Nevertheless, in 1934 Ladislas Morton succeeded in obtaining very crude (and still with less then optical resolution) images of a leaf sample. A considerably improved electron microscope, with at least an order of magnitude higher than optical resolution, enabled later to image viruses (at the time still somewhat “mysterious contagious entities”) as well as several bacteria, incl. the tuberculosis bacterium.

After the corrective optics was installed in 1993, the Hubble Space Telescope provided images without aberrations introduced by earth' atmosphere and with a quality unmatched by ground-based telescopes at the time (prior to adaptive optics), with much reduced background levels, sensitivity at all wavelengths, hight pointing stability, and wider field-of-view, which enabled numerous astronomical discoveries and improved our understanding of the universe.

Godfrey Hounsfield's first laboratory prototype of a X-ray CAT scanner, a tabletop-sized, rather crude and odd looking contraption to the naive observer, produced an image of a (preserved) human brain, which appeared to resemble a card from a Rorschach test instead of a CAT scan—yet it proved the fundamental physical principle (Hounsfield, Nobel lecture 1979). The first CAT scans of (live) human brains obtained 1972 at the Atkinson Morley hospital in London had such high noise levels and low spatial resolution that they had very limited clinical use. One of the original goals of this work was to detect, i.e., “see” (large) brain tumors against healthy brain tissue. Initially it was anything but clear if the required image quality in terms of signal-to-noise ratio, contrast, and resolution could ultimately be achieved. It took another decade to reach full clinical utility. It is in hindsight remarkable that during the late 70's and 80's the development of this technology received sufficient funding to move from a Nobel prize-winning curiosity to a $10 B industry today (for hardware alone).

All these problems were relatively easy to comprehend since they were essentially confined to a single domain (physics, mechanics, optics), i.e., inventors and instrument/machine builders and “early adopters” had similar technical and scientific backgrounds, thus understood the implications quickly. Nevertheless, many of the aforementioned breakthroughs as well as other inventions had “a difficult birth”. However, at the beginning of the 21st century we are beginning to see the emergence of a number of multi-disciplinary problems and technical challenges, where it is not instantly clear why progress has heretofore been slow. This is in particular the case for challenges related to the understanding of life, human health, molecular diagnostics, artificial intelligence, and even robotics. All these areas require knowledge in several areas including at least physics, electronics, optics, chemistry, biochemistry, medicine, neurology, computer science, and statistics.

One such example is the better understanding of life on a molecular level, in particular the understanding (and utilization thereof in clinically usable methods and devices) of the interplay of DNA (RNA) and proteins as well as the required instrumentation to perform measurements to gain such knowledge.

### Description of the Related Art

Modern DNA sequencing techniques require Polymerase Chain Reaction (PCR), i.e., the ability to chemically multiply (“amplify”) DNA (Mullis, Nobel lecture 1993). Only very recently have single-molecule sequencing machines become commercially available, which are not based on chemical amplification but on extremely sensitive physical detection methods (thanks to the fact that one can count individual photons). These machines represent a physical solution to a biochemical problem, but still only on the level of DNA.

Regardless which sequencing technology will eventually commercially prevail, few outsiders are aware of the limitations inherent in the analysis of DNA (and RNA). While clearly many highly interesting insights will be gained from inexpensive widespread DNA sequencing in future years, in particular related to molecular and evolutionary biology as well as the general probability (but not certainty) of an individual to eventually suffer from a certain disease. In fact, even this goal appears to be much more difficult to reach than anticipated since the search for such DNA based markers (i.e., direct links between DNA variants and the tendency to get a disease) has yielded surprisingly few results with sufficiently strong correlation (Hall, 2010, “Revolution Postponed”). Thus, the clinical application of DNA sequencing to medium term health problems is for very principle biochemical reasons limited if not questionable. Even if available, such probability statements based on detected DNA mutation can bring patients in a serious dilemma, having to decide if one should undergo (potentially unnecessary) preventive surgery. A recent example involving a prominent actress has been widely publicized.

Of much higher value would be the ability to detect the actually onset of a serious disease, or being able to predict the response to a drug, or to monitor in real time the response to a drug, and/or to choose or modify the composition of a drug in real time a based on measured data.

Life is largely based on protein chemistry. According to our current, likely incomplete understanding a 2% fraction of human DNA encodes for only about 22,000 proteins, although this number is still debated. Despite this seemingly small number, it is believed that the human body contains actually several millions of different versions of these proteins due to a) mRNA splice variants, and b) various reversible or irreversible chemical modifications (posttranslational modifications, PTMs) on proteins, which are critical for a person's health. The exact number is not (yet) known for the metrological reasons given below.

It is only through a better understanding of gene expression (i.e., protein biosynthesis via molecular transcription and translation), and of the protein networks, that deeper insights and ultimately truly predictive capabilities of human health and drug effectiveness can be potentially achieved. Mammalian cells produce over 1000 proteins (transcription factors, cofactors, and chromatin regulators), which play a role in gene expression, i.e., when and how much of other proteins are produced in the body (based on information residing in DNA), and if the process is performed without errors, yet our understanding of the involved processes and their dynamics is incomplete. Of particular importance in this context are, for example, histones and histone modifications. Thus, it is more accurate to imagine life as a dynamic interplay between DNA, RNA, and protein networks.

The abundance and composition of proteins in an organism (“intentionally” as well as due to errors in transcription and/or translation) is far more dynamic compared to changes of DNA during replication. Errors in biological information transfer from DNA to DNA (during cell division), and from DNA to proteins (via RNA) occur in cascades and at different time scales and with largely different probabilities.

If we ignore here extreme circumstances such as radiation exposure, the changes that DNA undergoes in an organism are orders of magnitude lower than those of proteins. It has been estimated that the error rates of DNA synthesis are about 10⁻¹⁰ to 10⁻⁸ (base pair mutations and frame shifts), of RNA synthesis on the order of 10⁻⁵, and of protein synthesis between 10⁻⁴ to 10⁻³. Notably, DNA replications utilizes a form of molecular proofreading and an error correction or “enzymatic editing” mechanism, without which DNA synthesis error rates would be on the order of 10⁻⁵ to 10⁻⁴ (which would make complex life forms effectively impossible). The correct function of this error correction mechanism, in turn performed by several proteins (e.g. ATR, Exo1, MutS, Msh2-Msh6, XPA, etc.), is thus essential for DNA synthesis. An interesting side aspect is the required technology to quantify such low rates and to detect ultra-rare DNA mutations.

Thus, the rates at which DNA modifications occur are several orders of magnitude lower than changes in (and on) proteins. Beside the impact of disordered proteins on cell-signaling, which may also eventually cause and/or indicate various diseases, of great practical importance are (accidental) changes in sequence, changes in abundance, and chemical modifications on proteins (PTMs). Beside errors during protein synthesis, proteins are constantly subjected to degenerative chemical influences, which can result e.g. in oxidation, glycoxidation, phosphorylation, deamidation, or other damages and modifications, and few repair mechanisms on proteins have thus far emerged. It is now in principle clearly understood that such chemical modification on proteins are associated with numerous disorders and diseases (e.g. Alzheimer's, Parkinson's, Huntington's, dementia, immune system diseases such as arthritis, cancer, and many more), since they affect countless pathways (including gene expression itself), but our knowledge of specific correlations is far from complete.

Furthermore, cells build proteins at a considerable dynamic range, and many particularly important proteins, such as transcription factors, occur at relatively low abundance. As a result, our understanding of gene expression through transcription and translation has come predominantly from studies conducted on purified molecules from large cell populations, which can obscure several aspects of this inherently “single molecule process”. While specific binding events can be detected on a single molecule level in individual cells, for example with optical detection, comprehensive analysis of protein expression at a single cell level (based on mass spectrometry as explained in more detail later) remains a highly desirable, but thus fare elusive goal, as discussed in more detail later.

The dynamic nature of proteins is furthermore illustrated by the concept of an “epigenetic landscape”, a term already coined in 1942 by Conrad Waddington. It is meant as a conceptual analog to illustrate the generally irreversible “decisions” a cell makes along its “trajectory” of molecular development (Goldberg, 2007). In the most general sense, epigenetics concerns the relationship between genotype and phenotype, i.e. how from the same DNA base pair sequence different protein expression level derive. As such, cell differentiation is at least in some respects an epigenetic process. A related concept is “phenotypic noise”.

In particular, epigenetic research concerns covalent and non-covalent modifications on DNA and on certain proteins, particularly on histones, and how such changes influence the function of chromatin. Since it is now believed that epigenetic changes are associated with several diseases, incl. at least some cancers, epigenetic mechanisms are now already being considered for therapeutic approaches. Again, the ultimate clinical application of such approaches will (also) depend on a better understanding of epigenetic regulators, including PTMs on certain proteins, which will require at least in part elements of the disclosed invention.

Thus, there is a heretofore unfulfilled need for innovation.

It is from a theoretical standpoint clearly understandable that the missing information is (thus far) hidden in protein abundance levels as well as in chemical modifications on proteins—posttranslational modifications—(and in some cases in part also in chemical modification on DNA/RNA. As already mentioned, the challenge when analyzing protein mixtures obtained from nature is twofold: complexity and dynamic range.

The dynamic range of proteins in bacteria is on the order of 10⁵, in human cells on the order of 10⁷ to 10⁸, and about 10¹² in human plasma (Anderson, 2002). In human plasma the 22 most abundant proteins account for about 99% of the protein mass (with serum albumin at ≈45 mg/ml) while information-carrying proteins incl. tissue leakage products, interleukins, cytokines etc. may occur at 1 . . . 10 pmol/ml (Anderson, 2002).

Due to the enormous complexity of human proteins (several millions) and their huge dynamic range, protein mixture analysis represents an extraordinarily complicated metrology problem.

Thus, the comprehensive determination of the composition of very complex protein mixtures, i.e., to measure how much and which proteins (including those with chemical modifications) are present in a sample, is the thus far unmet underlying technical requirement. This technical capability is the key to finding clinically usable protein biomarkers, from which various diagnostic tests e.g. for the onset, stage, or type of a specific disease, or for the expected or actual response to an administered drug can be derived.

As mentioned above, and to provide a historical analogy, one of the original motivations and goals behind the developments of X-ray scanners in the 1970's was to detect, i.e., “see” (large) brain tumors against healthy brain tissue. Initially it was anything but clear if the required image quality—today taken for granted—in terms of signal-to-noise ratio, contrast, and resolution could ultimately be achieved. In fact, the first CAT scans of (live) human brains obtained 1972 at the Atkinson Morley hospital in London had such high noise levels and low spatial resolution that they had very limited clinical use (Hounsfield, Nobel lecture 1979).

Like in the 70's during the development of CAT scanners, one of the goals of analyzing complex protein mixtures is to detect for example cancer at very early stage. But not as an image, rather by sensing molecules either directly in malignant cells taken during a biopsy, or molecules leaked from malignant cells into the blood stream, or molecules contained in malignant cells, which were released from a tumor and are circulating in blood. More precisely, by detecting minute abundance changes and chemical modifications on some proteins or peptides present in cells or in serum due to such leakage. The molecular pattern one is looking for is referred to as protein biomarker. However, despite the great potential, of such multi-parameter protein cancer biomarkers have not yet found their way into routine clinical application. Similarly, highly specific protein biomarkers would be instrumental as companion tests for certain drugs, which are only effective in a portion of the population and/or under certain condition. Without such tests, these drugs would never be approved for general use.

As a result, for more than a decade now, enormous efforts and substantial government and private funding (several billion dollars) were invested in attempts to find verifiable, highly specific and sensitive, clinically applicable protein biomarkers and to establish protein biomarker-based clinical diagnostics (Ahrens, 2010). The results are abysmal. A 2010 paper in Nature Biotechnology (Mitchell, 2010) brought the scope of the disaster to the attention of the scientific community: “By and large, the search for protein biomarkers—proteins that can indicate the presence of disease or how an individual is responding to therapy—has failed. Considering the hundreds of billions of dollars poured into proteomics research in the past decade, it is striking that not a single commercial molecule has emerged from it.” A similar article in Science “Digging for biomarker gold” summarized the problem as well (Harding, 2012). Likewise, an excellent BMC Medicine paper “The failure of protein cancer biomarkers to reach the clinic . . . ” points out various shortcomings associated with the clinical use of current insufficiently performing protein biomarkers (Diamandis, 2012).

In contrast to the disclosed invention, such early attempts of finding protein biomarkers used methods and shortcuts, which have at least in some cases hugely underestimated the vast gap between instrument performance and the demands posed by the complexity and dynamic range of serum, other bodily fluids, or even tissue samples (e.g. using a simple matrix assisted laser desorption ionization (MALDI) single MS linear time-of-flight (TOF) instrument and unfractionated or minimally fractionated serum samples). Thus, the required technical effort was misjudged or ignored. (By way of analogy, the resolution of the first electron microscopes built in the '30s and '40s were orders of magnitude too low to achieve atomic resolution. Any attempts to use these early microscopes e. g. to image crystallographic structures would have been bound to fail.)

Nevertheless, it is undisputed that the required information, i.e., highly specific and sensitive biomarkers, must reside in the protein expression levels (incl. potential PTMs). The technical challenge lies in obtaining the required information.

It shall also be remarked that thus far attempts using mRNA arrays (and to then infer protein abundance) have proven to yield also few if any useful results, since (a) mRNA levels correlate only weakly with protein expression levels and (b) PTMs of proteins can in principle not be detected this way. Thus the direct detection and quantification of proteins is required.

### SUMMARY OF THE INVENTION

For purposes of summarizing the invention and the advantages achieved over the prior art, certain objects and advantages of the invention are described herein. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

Disclosed are a rigorous method and apparatuses for the comprehensive analysis of complex mixtures of organic molecules, specifically biopolymers, in some embodiments predominantly mixtures of proteins, which in some embodiments ultimately serve to obtain the information constituting a biomarker, or similar biological signature, or to monitor health and ageing. In some embodiments such signatures or patterns may indicate the presence, stage, or type of a disease, the expected or actual response to drugs. In some other embodiments such a method and apparatuses may serve to monitor and/or control cell development. In some other embodiments such a method and apparatuses may serve to develop and use means to measure, influence, or control the speed or degree of aging of cells or organisms.

In some other embodiments such a method and apparatuses may serve to determine at least in part the nature of biological hazards, bioterrorism threats, or biological weapons, including those based on bacteria and viruses. In some other embodiments such a method and apparatuses may serve to select, develop, and or optimize countermeasures to biological hazards, bioterrorism threats, or biological weapons, including those based on bacteria and viruses.

In some embodiments the disclosed invention provides a method and apparatuses for the analysis of complex mixtures of predominantly biological molecules (incl. e.g. blood, saliva, sweat, sperm, cell(s), or samples derived there form) with respect to their protein content, the separation and fractionation of which in typically a plurality of dimension (of at least physical and/or chemical and/or biological properties) to enable at least one subsequent analytical method (in some embodiments comprising mass spectrometry), which determines with heretofore unrealized fidelity and degree of completeness (including exhaustively)
the composition of said proteins in said mix or sample, in terms of which kind proteins (amino acid sequences) are present, as well as
any previously known or unknown chemical modification thereon (including but not limited to what is currently referred as PTMs),
   present in biological regularly (“healthy”) or irregularly (“diseased”) functioning organism(s) or cell(s), and/or at least partially caused by intended or unintended exposure to or application of any form of treatment(s), drugs, any kind of conditioning, life style(s), work condition(s), or caused by ageing, cosmetics and/or other beauty products, medical procedures, surgical or other operations, predominantly physical processes, exposure to physical effects including but not limited to radiation, fields, pressure, furthermore exposure to or other intake of chemicals other than drugs, including but not limited to chemical additives in products, endocrine disruptors, air pollution, poisons, biological or chemical weapons, furthermore physical exercise, sport, sleep/rest, nutrition (incl. natural, processed, or man made), incl. contamination in or composition of food, or by coincidental or random effects (also in combination),
   any such said exposure, treatment, or influence, being known or unknown today, and furthermore in some embodiments also how many of each kind of protein (abundance) including how many (abundance) for each type of chemical modification.

All of these embodiments are intended to be within the scope of the invention herein disclosed. These and other embodiments of the present invention will become readily apparent to those skilled in the art from the following detailed description of the preferred embodiments having reference to the attached figures, the invention not being limited to any particular preferred embodiment(s) disclosed.

### BRIEF DESCRIPTION OF THE DRAWINGS

FIG. 1 illustrates highly schematically the process steps of the disclosed method. A raw sample 101, which may in some embodiments be derived e.g. from blood, saliva, other bodily fluids, tissue, cells, or otherwise, is optionally subjected to a preprocessing step 102, which may in some embodiments include, cleaning, filtering, freezing, thawing, extraction of cell content, removal or suppression of undesirable or meaningless (in the context of the specific investigation) content, addition of chemicals, or any other similar methods.

The resulting primary sample 103, containing at least a complex mixtures of organic molecules, usually biopolymers, and specifically in some embodiments protein mixtures, is henceforth referred to as the first plurality of sample molecules.

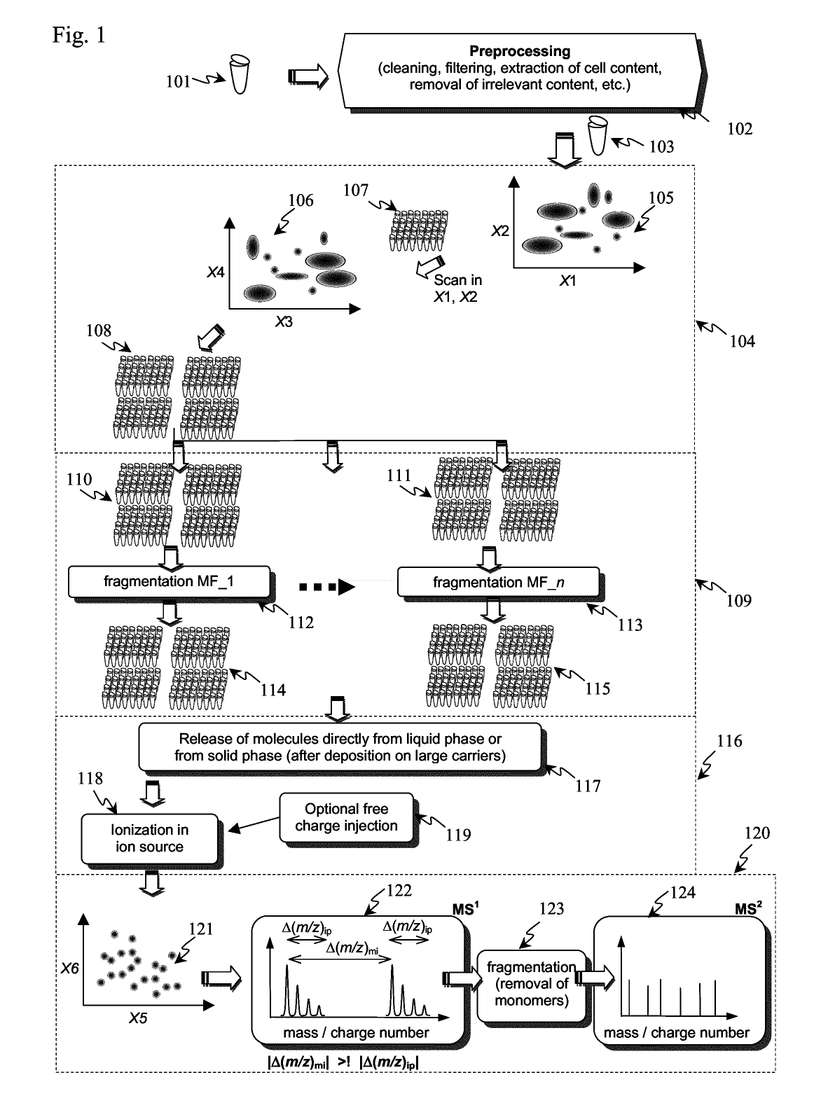

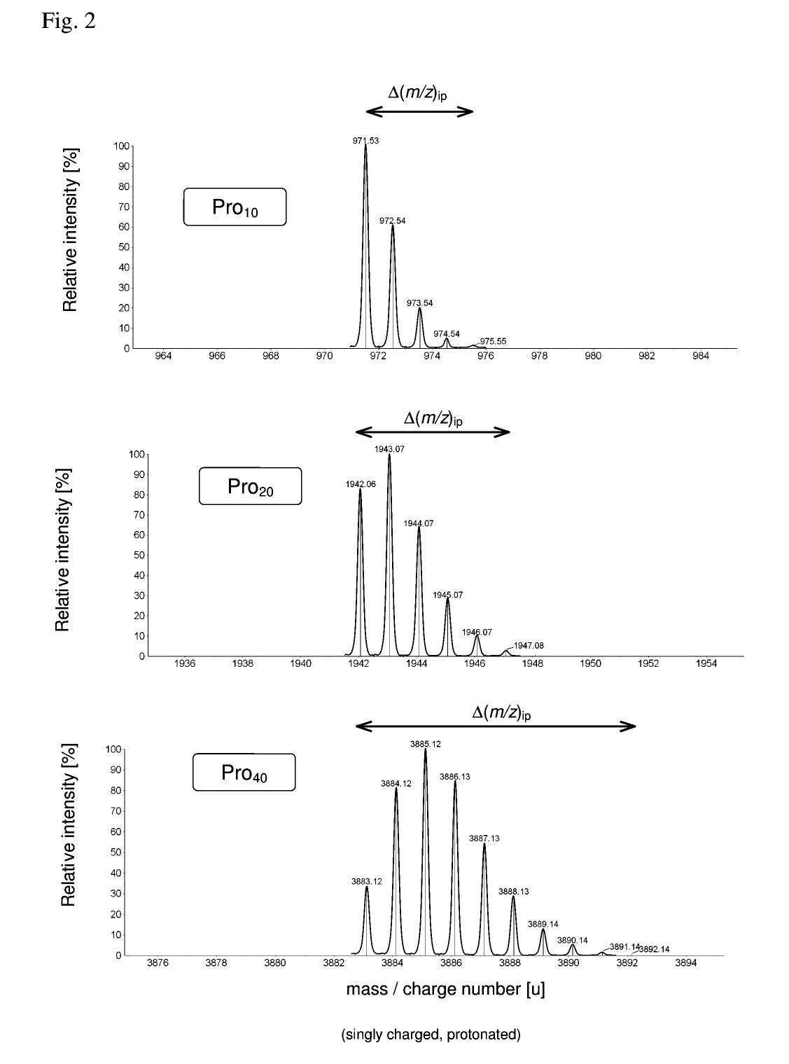

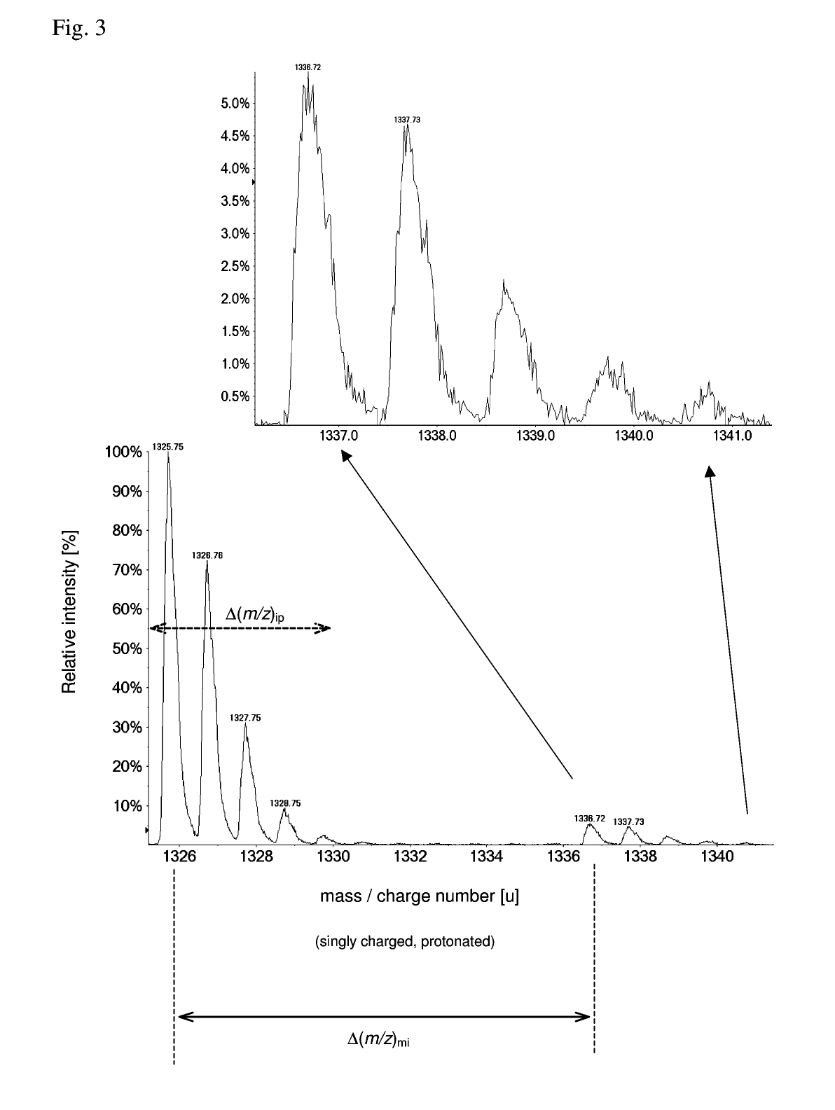

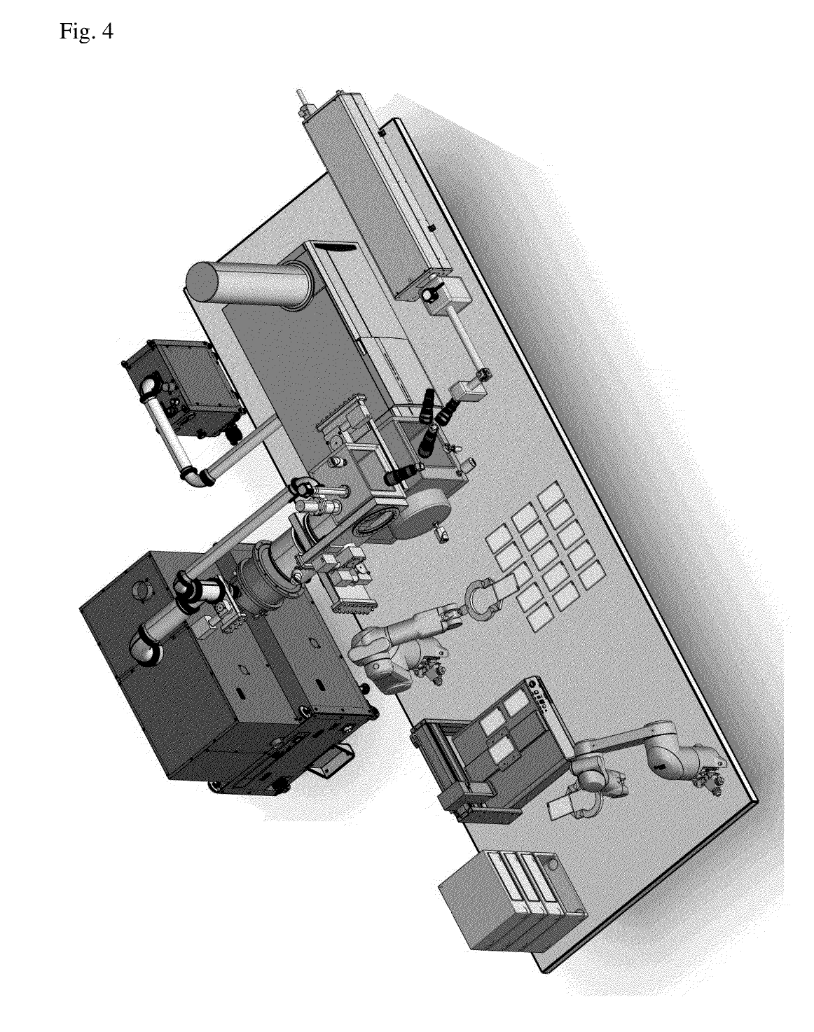

In a first process step 104 this first plurality of molecules is subjected to in general a multidimensional (in terms of chemical or physical properties) separation process. FIG. 1 illustrates an example wherein at least partially a 4-dimensional separation occurs, conducted in two consecutive steps 105 and 106, each of which separates at least partially in 2 dimensions predominantly simultaneously. The 4-dimensional parameter space is defined by parameters X1, X2, X3, and X4. First, the [X1,X2] sub-space is scanned, and then in [X3,X4] sub-space.

The latter is repeated several times and may be conducted e.g. for a plurality of points (“areas”, “subspaces”) in the [X1,X2] space; in some embodiments these scans are equidistant in the parameter space, and/or nonlinear, and/or discrete, and yet other only selected areas are picked.

Thus, in the shown example, the first scan produces a (discrete) two-dimensional plurality of samples 107, and the second scan produces a (discrete) four-dimensional plurality of samples 108. (This is illustrated here for simplicity as the most trivial case with two “data points” in each additional dimension X3 and X4, thus producing roughly four times the number of samples in plurality 108 compared to plurality 107. However, realistically each additional dimension may multiply the number of samples of the output plurality by a factor which may in some cases range from a few to a few hundred in order to achieve the required degree of separation.)

The individual samples contained in plurality 108 shall also be referred to as fractions.

As the next, second process step 109 at least one, but typically a plurality of physical and/or chemical molecular fragmentation steps are independently, put possible in parallel, performed, which break the biopolymers, specifically relatively large ones, in a controlled fashion into smaller pieces.

First, the plurality 108 is at least partially divided in to several pluralities, here labeled 110, and an unspecified number of additional aliquots up to those labeled 111.

The molecular fragmentation is in many embodiments done in a manner, whereby this process is selective to sequence information of biopolymers. Illustrated are molecular fragmentation sub-step “MF_1” 112 and an unspecified number of additional molecular fragmentation sub-steps up to “MF_n” 113. In many embodiments alternate fragmentation methods produce alternate fragments of the same biopolymer. However, at this stage the fragmentation methods do predominantly not break the biopolymers down to monomer level, they typically only reduce the molecular size (weight) to a range, which can at least in part be handled by the subsequent analytical process.

Said controlled fragmentation is performed independently (but typically in parallel) for each individual sample (i.e. for each members of pluralities 110 and 111), often contained in individual vials, wells, microtubes, or other cavities in large arrays etc. Thus, the number of input sample and output samples is approximately the same for each molecular fragmentation step.

The result, the second process generates third plurality of samples (114 and 115), the size of which depends on the size of plurality 108 (110 and 111 respectively, although the may each be smaller than 108), and the number of molecular fragmentation methods MF_n.

At least some members of said third plurality of samples (114 and 115), sometimes most if not all, are then subjected to a third process step 116. This is conducted for each member independently, but possibly in parallel on similar machines.

The now at least partially fragmented sample molecules are released in sub-step 117 either directly from predominantly a liquid, and/or in other embodiments they are first transferred to a predominantly liquid crystal, gel-like, or state solid state and then desorbed or otherwise released.

In many embodiments the latter is the case, since it helps to spatially and temporally decouple the previous steps from the following. Specifically, in some such embodiments addition molecules are added which may least in part support the process of desorption, protect sample molecules during desorption, and/or at least in part support or selectively support ionization.

The released sample molecules and/or fragments thereof are then ionized (although the release from the liquid or solid phase may occur in physical proximity and/or the same device or component of a device. This is in many embodiments a dedicated high performance ion source 118 for biological molecules. In some embodiments such an ion source may also employ additional free charge injection 119 to enhance ionization efficiency, enhance quantitative performance, and increase speed compared to conventional electrospray (ESI) and matrix assisted laser desorption/ionization (MALDI) ion sources. In some embodiments the charge injection also severs to produce sample ions with a specific, selected charge state.

In a subsequent fourth process step 120 said ionized molecules are then in some embodiments subjected to an additional separation process. Here illustrated is another two-dimensional process 121, which selects based on two additional properties. In some embodiments this may include ion collision cross section, symmetry, ion mobility, charge state, mass-to-charge ratio, dipole or higher moments, trajectory stability, or other physical properties, but in many embodiments related to the fact that sample molecules are at that point already ionized. In some cases this may include at least partially trapping said ions in suitable electric and/or magnetic fields.

Subsequently said ions are subjected to an analytical process, which determines at least one characteristic quantity of said ionized molecules, which is ultimately suitable to deduce at least the chemical composition of said ionized molecules, in many embodiments this will a the molecular charge-to-mass ratio, or molecular mass. Illustrated is such a first mass spectroscopic stage 122.

As pointed out in more detail further down, each (group) of molecular ions (of identical chemical composition) will produce a relatively wide comb-like pattern of peaks due to the distribution of naturally occurring isotopes, primarily ¹H, ²H, ¹²C, ¹³C, ¹⁴N, ¹⁵N, ¹⁶O, ¹⁷O, ¹⁸O, and others. For typical peptide masses the (only) monoisotopic peak is followed by several, sometimes 5 to 10 depending on the SNR of the spectrum and chemical composition of the peptide. The average width (mass range) of said isotopic pattern shall be denoted |Δ(m/z)ᵢₚ| and the average “distance” (or mass difference) between monoisotopic peaks (caused by molecular ions of different chemical composition) as |Δ(m/z)ₘᵢ|. A critical aspect of the disclosed invention is the requirement, to rigorously enforce that the degree of separation of all preceding separation dimension (stages), here X1 through X6, shall be such that on average the inequality |Δ(m/z)ᵢₚ|<|Δ(m/z)ₘᵢ| holds, and that in some embodiments |Δ(m/z)ₘᵢ| will actually be a multiple of |Δ(m/z)ᵢₚ|, i.e. |Δ(m/z)ᵢₚ|<<|Δ(m/z)ₘᵢ| to reduce the probability of overlap, since such overlaps would otherwise in most cases complicate or prevent detection and/or make unambiguous the subsequent sequence determination (incl. the determination of any chemical modification). One of the disclosed fundamental technical concept is that the smaller |Δ(m/z)ᵢₚ| is compared to |Δ(m/z)ₘᵢ|, the more likely is the successful execution of the entire process, i.e., the chance of finding a biological signature or similar insight.

In other words, the degree of separation must on average always be sufficiently high to ensure that during said at least one analytical process only a negligible degree of detrimental interference between said groups of essentially similar molecules or decomposition products thereof occurs with respect to obtaining said at least one characteristic quantity. This is in fact an extraordinary demand on the degree of separation, as eluded to further down.

In the illustrated example this first mass-spectroscopic sub-step is followed in many embodiments by at least one sub-step 123, which is based on one ore more physical and/or chemical methods to affect controlled fragmentation, which breaks off individual monomers, at least partially down to the monomer level (e.g. amino acids in case of proteins and nucleic acids in case of DNA and RNA). Typically various numbers of monomers are removed from the original molecular ion depending on chosen parameters of the used process. Thus again, a characteristic mix of complete and incomplete fragments is produced, from which sequence information can be obtained. (This fragmentation methods may include, but shall not be limited to collision induced dissociation, electron capture dissociation, electromagnetic radiation of sufficient quantum energy, or any other suitable method.)

In at least one subsequent sub-step 124, which determines at least one characteristic quantity of said fragments, from which ultimately the chemical composition can be deduced, the sequence information is derived, including any chemical modification on any monomer. Illustrated in FIG. 1 is another mass-spectrometric sub-step, referred to as MS-MS or MS².

FIG. 2 illustrated the increasing width of the isotopic pattern with increased molecular mass. Plotted are simulated spectra (based in the known isotopic distribution of elements) of 3 hypothetical peptides with different numbers of identical amino acids: Pro₁₀ (10 Prolines), Pro₂₀ (20 Prolines), and Pro₄₀ (40 Prolines), all assumed to be singly charges by protonation. Theoretical peak positions are drawn as infinitely “thin” lines (assuming infinite mass resolution. For illustration purposes only, typical curves have been graphically overlayed on some of the large peaks, to convey the appearance of a typical mass spectrum, assuming all peaks are resolved. (Realistically, the ideal infinitely thin peak would be convoluted by a function describing the mass resolution of an actual instrument, which can vary widely.)

FIG. 3 shows as a practical example a strongly zoomed-in portion of a mass spectrum of a tryptic digest of human histone H4 (a protein occurring in humans, which is of critical importance for DNA coiling and gene regulation), illustrating how closely spaced peaks may occur (even if there are only peptides from 1 protein in this particular sample and the protein is relatively small). The monoisotopic peak at m=1325.75 u corresponds to the [25-36] peptide sequence DNIQGITKPAIR (no missed cleavage), and the next monoisotopic peak at m=1336.72 u corresponds to the [45-46] peptide RISGLIYEETR (one missed cleavage, but of course usable for obtaining sequence information) and here occurring at a dynamic range of only 10:1. The mass difference between both monoisotopic peaks is Δ(m/z)ₘᵢ=10.97 u, which is just sufficient to enable unambiguous identification.

Each monoisotopic peak is followed by several isotopic peaks, which determine the total width of the isotopic pattern per fragment (here peptide), here approximately Δ(m/z)_{mi·}=5 u. If an unknown biopolymer (e.g. protein) mixtures are analyzed, overlapping of these peaks must predominantly be avoided because otherwise obtaining full and unambiguous sequence information (including any chemical modifications) without prior knowledge becomes effectively impossible, in particular also due to the typically much larger dynamic range. This illustrates again the need for said extremely high degree of separation (as well as very high SNR, specifically of the ion source) and the use if several different fragmentation methods in said second process step.

As pointed out above, said extreme demands in terms of separations will cause in all but the most trivial cases a very large number of measurements, which necessitate the use of automated and/or robotic high-throughput systems with strongly reduced signal acquisition time, which can perform the described process steps autonomously, uninterrupted continuous operation, and predominantly without user interventions.

FIG. 4 provides a rendering, which crudely depicts some of the elements, which may in some embodiments contribute to the required level of system performance. In many embodiments such systems will be centered around a high performance ion sources, since the ion source has critical impact on the entire system performance, namely sensitivity, speed (throughput), SNR, linearity, and quantitative performance.

In some embodiments custom-built ion sources may be connected to commercially available mass spectrometers. Here depicted, as one example, is a high-end triple-quad TOF MS. The relatively large main ion source chamber and the attached interface chamber (for sample carrier exchange) have in the depicted illustration large gate valves to enable the transfer of relatively large sample carriers.

Further, schematically rendered are a high power, highly stable, solid-state laser, typically with very high repetition rate, to support sample molecule desorption. Correspondingly strengthened laser beam delivery optics and computer controlled zoom, focus, and attenuation optics are partially illustrated. In some embodiments the laser beam itself is scanned two-dimensionally at a high rate. In other embodiments the beam (pulse) energy is sufficient a achieve at a laser spot size, which is of approximately the size of a sample spot, a flux sufficient to desorb individual sample spots without optical or mechanical scanning, solely by rapidly repeated pulse exposure.

Typically, 2D sample carrier movement/alignment and robotic sample carrier introduction/exchange are fully automatic for extended periods of time involving several sample carriers. In some embodiments sample spots desorption, ionization, analysis, and sample carriers and/or laser relocation to the next sample spot may occur in substantially less than one second.

In typical embodiments also high-resolution video image acquisition is used to monitor and in some embodiments to control the sample spot alignment and/or the desorption process. In such embodiments large windows, at least on one side of the ion source chamber, are used to enable optical access (video observation, laser input, input laser monitoring, reflected laser output, reflected laser beam monitoring, fluorescence monitoring, or any other secondary effect etc.). Typically the employed glass (quartz) is highly pure (low content of any contamination) and has low attenuation at a wide wavelength range, typically comprising UV and visible light, in some embodiments from UV to IR.

Furthermore, stylistically illustrated are (only a single) 2D-HPLC system and robot for sample handling, incl. digestion, and sample spotting on sample carriers. In typical embodiments this part of the setup will be more complex. Also illustrated are 2 6-axis robots for handling of microtiter plates and of sample carriers, incl. carrier insertion into and removal from the ion source.

While the illustrated sample carriers resemble 1536 spot plates, in typical embodiments these carriers will be larger and hold more sample spots. This enables to reduce the time penalty for sample carrier exchange (in and out of the ion source), which involves various sequential mechanical movements, as well as venting and pumping of various compartments, all of which requires time spans typically on the order of minutes, which are typically larger than the average spectrum acquisition time.

Much like throughput and efficiency of the microelectronics industry has benefited from using increasingly larger wafers (with more chips), so will the disclosed bio-analytical method benefit from very large sample carriers. In some embodiments such carriers may have a surface area (on which sample spots are deposited) on the order of several hundred cm², or even more. In some embodiments silicon wafers (optionally coated) will be used. In some embodiments such silicon wafers have functioning semiconductors on (in) them and/or metallic wiring, which is used to control and/or monitor aspects of the desorption and/or ionization, an/or analytical process.

Furthermore illustrated are additional vacuum components: A high-power turbo pump for the custom-built ion source, connected to a very large rough pump, as well as another additional (smaller) rough pump to support the rough vacuum generation for the mass spectrometer itself, since the ion source may transfer more gas into the mass spectrometer, than it was designed to handle.

In typical embodiments a comprehensive system for self-monitoring, self-protection (thermal, vacuum, electric, optics), as well as for protection of an operator or observer (e.g. against injury due to laser light or robots) is also employed. Numerous details incl. cables, fibers, temperature control, additional supporting structures, etc. are omitted in FIG. 4. Again, this illustration is to be interpreted only as a course conceptual depiction and many other arrangements at least in part based on some of the mentioned components will be apparent to those skilled in the art.

In particular, in some embodiments a relatively large plurality of separation sub-systems (performing process steps one and/or two) may produce prepared sample carriers, which are transferred to a smaller plurality of high performance ion sources and mass spectrometers.

In some embodiments a relatively small plurality of separation sub-systems (performing process steps one and/or two) may produce prepared sample carriers, which are transferred to a larger plurality of high performance ion sources and mass spectrometers.

In some embodiments a plurality of the disclosed predominantly automatic and/or robotic high-throughput systems for the analysis of mixtures of biopolymers will operate in parallel on one primary sample, in order to reduce the time for obtaining the desired information. In some embodiments a plurality of such pluralities may perform such task inside a large facility and do so at least in part as a service for clients, which submit samples for analysis.

### DETAILED DESCRIPTION OF THE INVENTION

As pointed out, the detection and identification of peptides and proteins faces one decisive problem: In contrast to DNA, there is no equivalent of PCR for proteins, i.e., proteins cannot be amplified chemically. Furthermore, proteins are more complex from a combinatorial standpoint (20 amino acids vs. 4 nucleic acids). This means that the sensitivity (and as a related parameter speed) has to come exclusively from the physical measurement.

In principle, it is possible to determine the amino acid sequence of a (small) protein purely by chemical and separation means, i.e., Edman degradation (1950) and Bergmann degradation (1932). In fact, the first full sequence of a protein was determined long before the first gene was sequenced: Frederick Sanger sequenced bovine insulin in two steps in 1951 and 1952 (Sanger, Nobel lecture 1958), whereas the first genome (of bacteriophage Phi X 174) was obtained in 1977, also by Sanger (Sager et al., 1977). However, such methods to identify proteins are highly laborious and slow, requires considerable amounts of a purified protein, and have several other limitation.

Thus, from a practical standpoint there are fundamentally only two ways of detecting and identifying proteins: (A) methods based on specific molecular binding events and (B) mass spectrometry (MS)-based methods.

Methods utilizing affinity-based specific molecular binding include any antibody-based methods—naturally occurring or synthesized—, but also including e.g. aptamer-based (i.e., synthetic) chip-mediated protein detection methods et cetera. Typical examples for antibody-based methods are enzyme-linked immunosorbent assays (ELISA), flow cytometry and variants thereof such as ‘mass cytometry’ (CyTOF)). Such methods can in some cases be highly sensitive, enabling single cell analysis in case of flow cytometry (ELISA is less sensitive). In many other case the affinity of certain antibodies is simply not high enough, e.g. for practical tests based on blood samples. In general, antibody-based methods are relatively cheap as long as one is only looking for a few proteins (thus one reveals only a tiny fraction of all proteins present in a cell). Nevertheless, the limited success of such highly restricted antibody based biomarker searches—based on information from very few protein levels—clearly indicates that understanding proteins is the key to clinically usable biomarkers.

However, comprehensive protein mixture analysis using methods based on specific molecular binding, including antibody-based methods, is (a) either physically impossible (in the case of flow cytometry due to wavelength overlap), (b) even if possible would be prohibitively expensive and/or impossible as it would require libraries of antibodies (or other molecules) against all known human proteins including posttranslational modification (PTMs, estimated on the order of several million) and (c) does not generally allow the clear identification and location (on which amino acid) of PTMs.

Thus, methods based on specific molecular binding, including antibody-based methods, are better suited for quick and cheap diagnostic tests, once one (or more) protein biomarker has already been identified (and such molecular binding are specific enough).

Conversely, at least in part on MS-based methods are, in principle, well suited for the discovery of biomarkers (based on the analysis of complex mixture), since they can detect any peptide and protein with any PTM. However, they are orders of magnitude less sensitive and intrinsically not quantitative (as a result of different proton affinities of amino acids). However, in order to obtain quantitative information from MS measurements investigations have resorted to complicated tagging methods (incl. isotope-coded affinity tags (ICAT), isobaric tags for relative and absolute quantitation (iTRAQ), tandem mass tags (TMT), metal-coded tags (MeCATs), and stable isotope labeling with amino acids in cell culture (SILAC) etc.). Unfortunately, this is either too expensive and time-consuming for comprehensive analysis, or brings one straight back to the problems associated with antibodies, or is not clinically applicable to humans.

Hence, in principle it is clear that mass-spectrometry based methods have the potential of finding markers or similar signature information. As eluded to above, previous attempts have failed for various reason, both biological as well as metrological, specifically approaches based on so called shotgun proteomics, whereby a primary sample containing a mix of proteins is first enzymatically digested, then fractionated, and the obtained fractions are then subjected to e.g. mass-spectrometric analysis. The problem with this approach is, if dealing with complex mixtures and little prior information about their composition, that some information about the composition of the contain proteins is lost, which would necessitate to unambiguously identify all protein fragments (peptides) and again fragments thereof (amino acids), including all potential chemical modifications (PTMs) of said proteins.

Said “shotgun” approaches may be suitable if a sufficiently high degree of prior information concerning the potential content of a sample, e.g. a protein mix is available, specifically if the goal of the analysis to confirm (or refute) the presence e.g. of specific proteins. Such statements can be made based on the detection of a few characteristic protein fragments (peptides), but no statements can be made about any other potentially present modifications on other fragments of such a protein. However, this is required for comprehensive biomarker searches in predominantly unknown protein mixtures.

Furthermore, previous biomarker searches have typically attempted to artificially reduce the number of mass-spectrometric measurements and/or degree of separation, with the fatal result of loosing large amount of information due to detrimental interference (in a general physical and or metrological sense, not a sense of wave physics) of signals within each measurement.

As a result, MS-based biomarker searches at present produce markers with insufficient sensitivity and specificity for general clinical application. What has been missing thus far is a truly comprehensive and sufficiently sensitive analysis of all proteins, preferably from very small amounts of tissue or serum, ideally even at the single cell level. Since no PCR exists for proteins, a physical solution to a biochemical problem is required. A critically limiting factor is the ion source performance and technology. A related factor is the degree of sample separation.

There are at least two fundamental technical parameters, which limit the amount of information—here typically the number of different peak position (by whatever definition)—which can be extracted from a (mass-) spectrum: Signal-to-noise ratio (and thus dynamic range) as well as resolution.

Every instrument, which determines molecular mass or mass-to-charge ratio, has a finite resolution, i.e. the ability to resolve different masses or mass-to-charge ratios. Thus, there is always a finite number of individual peaks which can unambiguously be identified within one spectrum and a specific problem associated with mass-spectrometric analysis of complex mixtures is the overlap of peaks, which in many cases results in the undetectability of smaller peaks (even if the signal-to-noise ration would be sufficient). It is critically important to recognize the implications of these facts.

Fundamentally, the disclosed solution to the problem of obtaining the thought information, e.g. for biomarkers in complex mixtures of organic molecules, specifically protein mixtures, is to combine
a first process step wherein a separation of said molecules occurs with respect to a single or plurality of one or both of physical or chemical properties producing a second plurality of samples, each containing a pluralities of organic molecules, which is less complex than said first plurality, and
in a second process step subjecting members of said second plurality of secondary samples to a processes wherein said organic molecules contained in said samples from said second plurality are at least in part fragmented into smaller molecular sub-units with lower molecular weight than the original molecules,
thus deriving from members of said second plurality of samples at least one new, third plurality of samples containing predominantly a larger number of groups of molecules with lower molecular weight compared to those produced after the first step, and
in a third process step subjecting samples from said third plurality of samples to a process whereby said contained organic molecules or fragmentation products thereof are released from a liquid, liquid crystal, gel-like, or solid phase, as well as ionized, and
in a fourth process step said ionized molecules are subjected to at least one analytical process (sometimes also at least another prior separation step), which determines at least one characteristic quantity of said ionized molecules, and
   wherein the degree of separation of said of said organic molecules, and of fragmentation products thereof, is sufficiently high to ensure that said samples belonging to said third plurality, contain few enough groups of essentially similar molecules or fragmentation products thereof,
   such that during said at least one analytical process only an acceptable (in some cases negligible) degree of detrimental interference between said groups of essentially similar molecules or fragmentation products thereof occurs with respect to obtaining said at least one characteristic quantity.

Detrimental interference shall mean any kind of detrimental effect on the ability to determine the said at least one characteristic quantity. Some examples this may entail any form of overloading of sensors or instruments, any form of exceeding a certain complexity of a sample (depending on the instrument used), likewise exceeding a certain dynamic range in/of a sample, in some embodiments this may specifically be caused by the overlap of peaks in mass spectra, and/or the overlap of isotopic envelopes in mass spectra.

Said separation(s) of said molecules during said first process step consists in many embodiments of a plurality of dimensions, whereby such separations may occur simultaneously and/or sequentially. Dimension shall refer to at least in part distinguishable physical and or chemical properties of said molecules (or fragments thereof), such as mass, mass-to-charge ratio, (collisional) cross section, physical cross section, any form of mobility in a medium or in a field, the specificity, or, likelihood, or reaction rate of specifically binding to other molecule(s), isotopic composition, spin, charge state, net charge, electric dipole moment, electric quadrupole moment, any higher electric moment, magnetic moment, relative or absolute permittivity, relative or absolute permeability, magnetic susceptibility, electric conductivity, any vibrational mode(s), any other stable or excited energy level(s), translational or vibrational energy (“Temperature”), times to relax from excited modes, et cetera.

In some embodiments this may include any form of n-dimensional HPLC, other chromatography, electrophoresis, free-flow electrophoresis, or any separation with a resting or flowing medium, with a controlled flow profile, in some embodiments at least in part also under the influence of electric fields.

In some embodiments said process step one may include any form of specifically temporarily or permanently binding sample molecules to other molecules, including but not limited to antibodies and aptamers (including optionally tagged versions thereof), either in solution in volume, or on surfaces. In some embodiments this may result in surfaces covered with the at least partially separated sample molecules, and in some embodiments said second process step of fragmenting said sample molecules into smaller units (e.g. by digestion or otherwise) may be conducted directly on said sample molecules attached to said surfaces. In some embodiments these surfaces may subsequently be subjected to said process step three and four. In some specific embodiments that entails mass spectrometric analysis of said smaller molecules obtained in process step two, or at least fragments of said molecule (including down to individual atoms).

Thereby the original primary and very complex sample is separated in a very large plurality of chemically essentially similar or identical molecules, e.g. a few proteins. In some embodiment this may only be a single protein, but some of the members of the separated ensemble may have PTMs. In some other embodiments this may be a small number of proteins, typically a single digit number.

In many embodiments said multi-dimensional separation step(s) will not be able to separate molecules of identical chemistry but different isotopic composition, i.e. they will not separate molecules which only differ in the number of contained neutrons (but have identical amounts of protons and electrons). This in turn implies, that each fragment of such a molecule, subsequently produced during the next process step, occupies a relatively large mass range during e.g. a mass-spectrometric analysis, performed in yet another subsequent process, due to its natural isotopic pattern (as a result of naturally occurring distribution of isotopes, namely ¹H, ²H, ¹²C, ¹³C, ¹⁴N, ¹⁵N, ¹⁶O, ¹⁷O, ¹⁸O, and others).

For the majority of molecular fragments, present in a single (mass-spectroscopic) measurement, any spectral overlap must be avoided. This is one of the primary reasons why the disclosed method of analyzing complex mixtures of organic molecules, such as proteins, places such high demands on the degree of separation.

During the next, second process step the molecules, contained in each fraction said of second plurality of samples, produced (“fractionated”) during said first process step, are fragmented. This intentional and controlled fragmentation during said second process step, of the now at least partially separated molecules from said primary sample, shall include any method or process by which said organic molecules, which were contained in the primary sample, are at least partially split into smaller molecular units, including any suitable chemical and or physical methods, such as enzymatic digestions, chemical or physical dissociations, collisions, collisions with charged or neutral atoms, molecules, molecular clusters, or other particles; exposure to electric fields, electromagnetic waves, microwaves, X-rays, or generally radiation or photons from various sources or devices including but not limited to UV, optical, and IR lasers, X-ray lasers, free electron lasers, or any other form of bond breaking.

In some embodiments said second plurality of samples, produced (“fractionated”) during said first process step, may at least partially be divided into two or more sets of secondary samples (smaller in terms of each volume) prior to said fragmentation. These are then subjected to different types of fragmentation, such as different enzymatic or other chemical digestions, which tend to produce different fragments (peptides), since they preferentially break bonds at certain sequences (of the underlying units, such as amino acids), thereby increasing the probability of full peptide coverage for the majority of proteins in the primary sample.

Subsequently at least some, but many embodiments the majority of the thereby produced third plurality of samples is either desorbed directly from a liquid phase (of a solution or buffer in which they are contained), or are placed on surfaces, typically as a relatively large number of drops, and in some such embodiments additional molecular components may be added prior or during the deposition of said drops on said surfaced. In some embodiments said additional molecular components may serve to protect said sample molecules during a subsequent desorption event, may at least in part support the subsequent ionization, or may at least in part support the preservation of said samples over an extended period of time.

In some embodiments the release and ionization of sample molecules may be performed by conventional Electrospray or MALDI ion sources. In some embodiments the performance of such ion sources may be insufficient or at least detrimental to achieve the desired results of finding certain signatures or biomarkers since errors introduced during the release and ionization of said sample molecules are in most cases not correctable, both physically and/or subsequently numerically.

To use an analogy, ion sources for biomolecules are to a mass spectrometer, what lenses are to cameras. One conditions and prepares biomolecules, the other photons for further analysis. The most expensive camera with the largest sensor is of little use, if a cheap lens distorts the image and its small aperture permits only few photons to pass through.

Such shortcomings concern primarily signal-to-noise ratio, background “signal” (unintentional) molecular fragmentation, interaction with embedding molecules incl. formation of adducts, tendency to produce ions of different charge state (i.e., some sample molecules undergo simple protonation whereas other carry multiple protons) which fills the spectrum further (while providing little or no additional information and which may required signal deconvolution, invoking well known numerical problem), general lack of sensitivity, different sensitivity for different sample component (in many cases due to different proton affinities), charge competition effects between sample components (i.e., the sensitivity e.g. for one specific peptide depends on the abundance of another present peptide), insufficient speed, and nonlinearity.

In other embodiments these may be modified, derived, or advanced forms of such conventional ion sources. In yet other embodiments such ion sources may be based on yet different or modified chemical and/or physical principles. In some embodiments such ion sources may at least in part be based on advanced collisional ion cooling schemes and/or electro-pneumatic superposition.

In some embodiments such ion sources may at least in part be based charge injection, whereby relatively small (compared to the typical fragment size) ions, which are separately produced, are brought in contact with the desorbed or otherwise from a solid or liquid (incl. liquid crystal and gel-like) phase released sample molecules, thus at least in part causing at least some of said sample molecules, or at least parts thereof, to be ionized.

In some such embodiments the injected ions are derived from atoms, which were ionized by removal of one or more electrons. In some such embodiments the injected ions thus carry multiple positive charges, according to the number of removed electrons. Thus, in terms of their effect of subsequently ionizing sample molecules, at least some of said injected ions, for example Na⁴⁺ or K⁵⁺, can be imagined as “multiple protons”, i.e., they carry multiple positive charges, but of course their atomic mass is also notably larger than the mass of the corresponding number of protons, in this example four or five protons. However, that additional mass (added to the ionized sample molecule) can be easily corrected for in the subsequent at least one analytical process, just like the added mass from ionization by protonation from a single proton is corrected. Moreover, in terms of their effect of subsequently ionizing sample molecules, at least some of said injected ions can be imagined as “super protons”, i.e., they not only carry multiple positive charges, but all electrons have been stripped from them, i.e., they are bare nuclei, for example Li⁺³ or Na⁺¹¹. The atomic mass of these injected “super protons” (i.e., bare nuclei) is in general nevertheless larger than that of the corresponding number of protons since of course said nuclei will also contain neutrons; in the given example 4 neutrons for the naturally most abundant ⁷Li and 12 neutrons for ²³Na, resulting in a total mass of the nuclei of approximately 7 u and 23 u respectively. But again, that additional mass (added to the sample molecule) can be easily corrected for in the subsequent at least one analytical process, just like the added mass from ionization by protonation from a single proton is corrected. One of the advantages of using such bare nuclei is that they may be highly reactive. Another advantage is, that using ionization by such bare nuclei or even by said multiply charged ions will shift the mass-to-charge ratio of an ionized sample molecule to lower values compared to ionization by single protonation. These arguments apply to most if not all producable multiply charged ions and nuclei used for injection.

In some embodiments such the high-performance ion sources may be custom-built and connected to commercial mass spectrometer.

In some embodiments the injection of said relatively low molecular weight ions, which at least in part serve to ionize said organic molecules and or fragmentation products thereof, is performed in such a manner that also predominantly quantitative information about the amount of said organic molecules and or fragmentation products thereof present is a sample, can be derived within the subsequent fourth process step.

In some embodiment said injected relatively low molecular weight ions comprise at least some, which carry multiples of unit charges.

In a subsequent process step the ionized sample molecules are introduced in an analytical instrument, in many cases at least capable of determining molecular mass-to-charge ratio or molecular mass.

As mentioned, said determination of the molecular mass or mass-to-charge ratio, has a finite relative resolution, i.e. the ability to resolve different masses or mass-to-charge ratios (defined as ratio of total mass over absolute resolved mass). Depending on the employed ion-optical configuration and built quality of the specific instrument, values may range from a 10² (very poor performance) to about 10⁶ (superb performance, in future perhaps even more), but they are of course always finite.

Simultaneously, one has to consider the impact of the above mentioned isotopic patterns produced by each chemically identical group of sample molecules. The “size” or “width” (i.e., mass range) of such isotopic pattern depends on the size (mass) of the molecule (or fragment thereof).

For example, as a rule of thumb, a tryptic digest of a not untypical protein with about 60,000 u mass will at least create roughly 20 theoretically detectable peptides within a mass range between m=1000 to 5000 u (singly charged), in many cases more due to incomplete fragmentation. Each peptide will, beside the monoisotopic peak, produce at least 5 to 10 adjacent non-monoisotopic =‘isotopic’ peaks. In other words said width of said isotopic pattern is several mass units, about 5 to 10 u.

In order to maximize the probability for unambiguous and comprehensive (nearly full peptide coverage) of all proteins contained in the primary sample (protein mix) we require that the probability for those sets of peaks (per peptide) to overlap is small. Thus, said degree of separation and number of dimensions of said separation must be extremely high, in some cases requiring separation down to the level of individual proteins or even beyond (i.e. the by fractionation isolated and then fragmented (e.g. digested) protein is further separated in said ion mobility stage prior to the actual determination of the mass-to-charge ratios)

A practical example is illustrated in FIG. 3, which shoes a strongly zoomed-in portion of a mass spectrum of a tryptic digest of human histone H4, with two valid isotopic patterns only about Δm=11 u apart. Each monoisotopic peak is followed by several isotopic peaks, which determine the total width of the isotopic pattern per fragment (peptide). If unknown, digested protein (mixtures) are analyzed, overlapping of these peaks should be avoided because otherwise unambiguous identification without prior knowledge becomes effectively impossible, in particular also due to the usually large dynamic range.

Therefore, the monoisotopic peaks need to be separated (again statistically on average) typically a multiple of the average width of the isotopic pattern. If, for example, one would require that on average the ‘distance’ be about 40 u, then each spectrum taken of a mass range from m=1000 u to m=5000 u should not contain more than (5000 u−1000 u)/40=10² monoisotopic peaks (which still produces >600 isotopic peaks in the same spectrum, given sufficient mass resolution). Of course the actual position of the monoisotopic peaks depends on the fragmentation method(s), e.g. type of digest or otherwise, and the sequence of building blocks (e.g. amino acids).

{If the mass resolution, in particular at higher masses, is insufficient to resolve isotopic peaks one is forced to deal with very broad peaks and their average masses and above made arguments about peak separation fully applies as well. If, however, in future suitable mass spectrometer with far higher resolution should be available (in absolute peak width of a monoisotopic peak substantially less than 0.1 u), then some of these requirements may in some cases be somewhat relaxed (but not by orders of magnitude) because it may be possible to assign individual isotopic peaks to the corresponding monoisotopic peak even if these ‘peak bundles’ as a whole overlap, i.e. the appear as a comb inside another comb.}

Thus, in some embodiments there is at least one additional separation step in at least one additional dimension, performed prior to the determination of the molecular mass-to-charge ratio or molecular mass using the already ionized sample molecules or fragments thereof (specifically in case of large proteins, which produced many fragments, thus potentially too much for a single MS spectrum). In many such embodiments such additional separations steps are based on ion mobility in gases under the influence of at least partially and/or temporarily applied electric field, which may have in general an arbitrary, but predominantly controlled spatial distribution and time-dependence. In some application at least some of said sample ions may be trapped by suitable electric and/or magnetic fields prior to the determination of the molecular mass-to-charge ratio or molecular mass.

In many embodiments, following said first level of mass-spectrometric measurements, there may be an additional, intentional fragmentation step, performed on said sample ions (i.e., typically peptide ions) and typically performed on individually selected sample ions (i.e., typically peptide ions), followed by at least one additional level of mass-spectrometric measurements performed for each of said selected ions, resulting in predominantly complete sequence information (including any PTMs) of said selected ion. In typical embodiments where predominantly protein mixtures are analyzed, the amino-acid sequence (and any PTMs thereon) of the selected peptide is determined. In some other embodiments DNA and/or RNA may be analyzed and in above mentioned step predominantly nucleic acid sequences of DNA and/or RNA fragments are determent, including any chemical modification thereon, including any molecules only weakly bound to said nucleic acids. Commonly this process often referred to as MS-MS, or MSⁿ in case of an arbitrary number of MS dimensions.

The above explained, the required very high degree of separation results in a correspondingly high number of required individual measurements. For most practical embodiments this will necessitate the disclosed use at least partially autonomous systems, including robotic systems, which are performing these and other related process steps.

The order of magnitude of the problem shall be illustrated with the following example. The before-mentioned enormous complexity of protein samples, e.g. derived from human plasma, in conjunction with the finite mass resolution of even the best mass spectrometers necessitates a very high degree of sample separation (in as many as possible dimension) to ensure that only a small number of proteins (digests) ends up in a single mass spectrum in order to enable unambiguous and complete detection of all proteins and any potentially present PTM.

For argument's sake it will be assumed here, that a relatively large number of on average 10 proteins is allowed in each sample from said second plurality. Thus, further assuming only a single fragmentation method (e.g. digestion) and assuming 20 peptides per protein, roughly 200 monoisotopic peaks) and correspondingly on average 200 required MS-MS spectra per secondary sample.

As mentioned, this number is far greater than what is normally required to identify a previously known protein from a number of peptides. However, in this application we must (a) assume that potentially all peptides may contain PTMs, and (b) thus strive to have a system which could potentially cope with the requirement of enabling full coverage, i.e., all peptides of every protein in the mix are potentially subjected to MS-MS to identify and locate PTMs. This also implies that, realistically, more than one kind of digest is required to increase the probability of full peptide coverage.

An example shall be provided. The disclosed invention is in some embodiments in particular useful to analyze complex protein mixtures. (For this estimate protein A and protein B having the same amino acid sequence but different PTMs shall count as different proteins.) For example, if one wishes to completely analyze 10⁵ proteins in samples (e.g. derived from a drop of blood) one would in some embodiments need to separate the sample in 10⁴ fractions and thus acquire 10⁴ single MS spectra and 200·10⁴=2·10⁶ MS-MS spectra on a single biological sample.

To conduct a study on two cohorts of 2·100 patients (e.g. one healthy and one with a certain disease, or one given a certain drug and the other given a placebo) would thus require

200·10⁴=2·10⁶ single MS spectra and

200·2·10⁶=4·10⁸ MS-MS spectra.

(This is the worst-case scenario. Smart algorithms may reduce this number by avoiding MS-MS on peptides with no PTMs.)

According to the disclosed invention a high-throughput systems are used for the analysis of the sample containing said complex mixture, which are predominantly autonomous and or robotic, continuously operating and which, in some embodiments, specifically using said charge injection, do not require tagging of any kind (i.e., no antibodies), and which can acquire a single MS (or MS-MS) spectrum with high SNR and in orders of magnitude shorter amounts of time than currently possible (requiring correspondingly higher sensitivity).

For example, if such a system requires 0.1 second per MS measurement, it can acquire the above mentioned 2·10⁶ single MS spectra in about 2.3 days and the 4·10⁸ MS-MS spectra in about 463 days. Since the problem can be parallelized, a battery of 10 such systems would be able to acquired all spectra in about 46 days, which is a practically acceptable time frame. (Smart control algorithms can also reduce the number of required MS-MS spectra if no PTMs are detected.)

In some embodiments such predominantly autonomous and/or robotic systems will have the size of a small room and will be centered around advanced high performance ion sources, high-end mass spectrometers, as well as robotic sample preparation and separation systems. At the beginning a user would simply insert a biological sample and after several hours or days could “pull out hard discs(s)” with the acquired data (or have the data transferred over a suitable network).

The amount of acquired raw (uncompressed) data is actually substantial in such a case. Assuming that the data amount for a single raw MS spectrum is 2 M Byte and for a single raw MS-MS spectrum 100 k Byte, then the 4·10⁸ MS-MS spectra would correspond to

$$\begin{matrix} & 2 \cdot {10}^{6} \cdot 2 \cdot {10}^{6} = 4 \cdot {10}^{12} = 4 T Byte \\ + & {10}^{5} \cdot 4 \cdot {10}^{8} = 8 \cdot {10}^{13} = 40 T Byte \\ & = 44 T Byte .\end{matrix}$$

Realistically, not one but several fragmentation methods must be used in the second process step, in order to obtain full sequence information, and the above estimated effort in terms of required time and data multiplies accordingly.

Thus far, such comprehensive and rigorous studies have not been conducted (at least in cases where the ultimate goal is clinical application). The failure of the entire proteomics “industry” during the last 15 years to discover clinically useful biomarkers can in part be understood in this light. We have only scratched the surface of proteomics in terms of comprehensive and quantitative analysis.

Only with the disclosed methods and the resulting level of comprehensiveness will there be realistic chances to routinely find clinically usable protein biomarkers (and understand their biological meaning).

Secondly, similar to the foregoing example, which illustrates the use of the disclosed invention for extremely high throughput and in some embodiments intrinsically quantitative MS measurements for comprehensive analysis of highly complex protein mixtures, such as plasma (e.g. for biomarker), the disclosed invention is in some embodiments in particular useful to (sometimes comprehensively) analyze relatively “simple” objects, such as individual cells, from a relatively large number of populations. Such a scenario emerges when analyzing e.g. intratumor heterogeneity. For example, an individual macroscopic tumor will in fact typically consist of a population of different cell types, or subclones, which may not only be characterized by (relatively small differences in DNA), but more importantly by differences in protein abundance and PTMs thereon.

In many cases conventional chemo-therapy subjects this population to selective pressure, with the frequent result that initially a tumor shrinks, since the weakest member of the pool die, but the treatment-resistant tumor cell types will survive, metastasize, and eventually kill the patient. Thus, again, deeper insight in the role of proteins and possible approaches for advanced forms of treatment would require the analysis of e.g. “only” 1000 proteins from per cell, but taken from 100 different cell types, which, in terms of complexity and throughput, brings one right back to the above discussed cased with 10⁵ proteins in a sample. Thus the disclosed invention can in some embodiments be used (a) to better understand tumor heterogeneity, (b) to tailor and/or select drugs based on (c) the individual person or animal, which has the tumor, and/or based on (d) the specific structure of the subclonal population. Furthermore, the disclosed invention may in some embodiments be used to dynamically monitor the response of a patient to treatment (i.e., observing changes in said subclonal populations) and to adjust the administered drug(s) including in terms of dose, composition, how frequently they are given etc. in order to achieve one or more goal of said treatment.

Thirdly, the disclosed invention will in some embodiments be useful to support the increase of our understanding (and potentially manipulation) of stem cell development and cell differentiation, i.e. how and why a cell ‘chooses’ to express a certain set of proteins, which is a subset of all the possible proteins (encoded in its DNA), and which remains to be a scientifically highly desirable, yet a thus far unattained goal. The ramifications for basic science would be profound and possible clinical applications far reaching (regenerative medicine; growth of tissue and organs from one's own body etc.).

To partially or comprehensively observe and to thereby potentially completely understand cell differentiation, requires to follow cells over several generations, while obtaining ideally complete information of which proteins are present in these cell, including all PTMs, and at which amount. Again, the disclosed invention may in some embodiments be used to perform some of the required measurements, in some embodiments comprehensively, and in some embodiments, (specifically those with charge injection) also with ultra-high sensitivity and intrinsically quantitative.

In some embodiments of the disclosed invention such high-throughput analysis of protein mixtures, derived from individual or a few stem cells, or from cells during the process of differentiation, will serve to obtain information which
(a) explains the process of cell differentiation, and/or
(b) allows to derive method to correct errors during differentiation (incl. drugs, enzymes, other proteins, peptides, amino acids, or other chemicals, which may be added or removed to correct at least in part such errors), and/or
(c) enables to derive and or control method to reverse the process of cell differentiation (incl. drugs, enzymes, other proteins, peptides, amino acids, or other chemicals, which may be added or removed to induce or support such reversal), and/or
(d) enables to derive method to induce the process of cell differentiation in a specific direction, (incl. drugs, enzymes, other proteins, transcription factors, peptides, amino acids, or other chemicals, which may be added or removed to induce or support such reversal, i.e. to produce e.g. induced pluripotent or multipotent stem cells), i.e., to produce cells of a specific desired type or types, in some embodiments based on already differentiated cells previously taken from the same person or animal, and in some embodiments to then grow tissue or organs from said cells with induced differentiation.

Fourthly, the disclosed invention will in some embodiments be useful to obtain information to better understand the ageing of higher eukaryotes, in particular of humans. In some embodiments the disclosed invention can be used to derive methods or to monitor and/or control methods to slow down or the reverse the effects of aging ageing of higher eukaryotes, in particular of humans.

A critical factor associated with the progression of aging are changes in protein synthesis and protein “quality control”, and the question why cells eventually loose the ability to perform these processes with sufficient fidelity. Thus, again, the key to practical applications will be the ability to study protein expression levels on a large scale, and in an ultra-sensitive, intrinsically quantitative manner, and based on mass spectrometry, as enabled by the disclosed invention.

There are Several Embodiments of the Disclosed Invention Analyze Complex Mixtures Organic Molecules, Specifically of Polymers, Regarding the Type and Sequence of Conducted Separation and Analytical Steps as Well as with Respect to Other Related Aspects:

In some embodiments said degree of separation being high enough such that for the majority of ions associated with a particular organic molecule and or fragmentation products thereof, and simultaneously acquired during one measurement constituting said analytical process, the isotopic envelopes do not overlap.

In some embodiments, in particular when said primary samples contain biopolymers, including but not limited to proteins, polypeptides, DNA, and or RNA, said analytical process may further comprise
a first level of one or more measurements conducted on said primary ions, and
at least one additional level of essentially similar measurements, which are being conducted on at least some of the groups of ions constituting any one of the primary peak during said first set of measurement, and
wherein between said first level of measurements and said at least one additional level of measurements at least some of those ions, which are subjected to said at least one additional level of measurements, are being fragmented in a controlled manner suitable to derive addition information about one or both of their chemical composition or sequence information in case of biopolymers during said at least one additional level of measurements.

Specifically, in some embodiments said at least one analytical process serves to at least in part determine any single or any combination of
an amino acid sequence, and
any chemical modification on any such amino acid, and
any other molecule, weakly bound to any such amino acid.

Specifically, in some embodiments said at least one analytical process serves to at least in part determine any single or any combination of
a nucleic acid sequence, and
any chemical modification on any such nucleic acid, and
any other molecule, weakly bound to any such nucleic acid.

In some embodiments said separation in said first step is at least in part being based on separating molecules in at least one liquid medium and at least partially under the influence of an electric field.

In some embodiments said at least one liquid medium is being controlled in such a manner that a flow profile is established, which supports said separation during said first step.

In some embodiments said second step, wherein said organic molecules contained in said samples from said second plurality are at least in part being decomposed into smaller molecules, at least in part by one type or a plurality of types of enzymatic digestions.

Some embodiments comprise a single or a plurality of measurement setups, which are predominantly capable of performing said process steps, and wherein one or both of predominantly autonomously operating or predominantly robotic machines are performing any one or any combination of said first, second, third, and forth process steps.

Robotic machines shall include but not be limited to machines, for example, which are typically computer controlled (including systems based on hardware or software implementations of artificial neural networks, and or artificial intelligence based systems), and which are typically powered by electricity, and which enable a plurality degrees of freedom of at least one mechanical motion or action (such as grabbing, moving objects, inserting objects into other objects, arranging objects, placing drops of liquids into volumes or on surfaces etc.). In some embodiments such robotic machines may resemble human arms and or hands and in other embodiments such machines may be able to perform motions, which are even more complex than what a human operator could perform.

Some embodiments comprise one or both of predominantly autonomously operating or predominantly robotic machines taking at least in part fractions of said primary sample, obtained during said first process step, and placing them in a plurality of volumes or a plurality of surface spots.

Some embodiments comprise one or both of predominantly autonomously operating or predominantly robotic machines taking at least in part fractions of said primary sample, obtained during said first process step, and placing them in a plurality of volumes or surface spots, thus at least in part establishing said second plurality of samples.

Some embodiments comprise robots subjecting at least in part said second plurality of samples to at least one type of enzymatic digestion thus at least in part establishing said third plurality of samples.

Some embodiments comprise one or both of predominantly autonomously operating or predominantly robotic machines taking at least in part samples from said third plurality of samples and placing at least parts thereof on carriers, which are subsequently introduced into and later removed from said at least one instrument, which is performing at least in part said fourth process step.

Some embodiments comprise one or both of predominantly autonomously operating or predominantly robotic machines placing said sample carriers into said at least one instrument, which is performing at least in part said fourth process step, and removing them from it.

Some embodiments comprise a plurality of separation systems and robots producing said third plurality of samples, which are being placed on said carriers, and introduced by one or both of predominantly autonomously operating or predominantly robotic machines into a single analytical instrument, which at least in part performs said fourth process step.

Some embodiments comprise a single separation system and robots producing said third plurality of samples, which are being placed on said carriers, and introduced by robots into a plurality of analytical instruments, which at least in part perform said fourth process step.

Some embodiments comprise connecting one or a plurality of computers, which are at least in part controlling one or a plurality of analytical instruments, which are at least in part performing said analytical process during said fourth process step, to at least one other computer, which is aggregating data representing at least some of said characteristic quantity determined by said at least one analytical instruments.

There are Several Other Specific Embodiments and Applications of the Disclosed Invention to Analyze Complex Mixtures Organic Molecules, Specifically of Biopolymers:

Some embodiments comprise conducting said process steps on a plurality of primary samples, taken from or derived from a plurality of individual organisms, and deriving at least in part from the thereby obtained data the information for a biomarker, which at least in part can indicate the onset, or stage, or type of a disease.

Some embodiments comprise conducting said process steps on a plurality of primary samples, taken from or derived from a plurality of individual organisms, and deriving at least in part from the thereby obtained data the information for a biomarker, which at least in part can predict the expected efficacy of a drug or mix of drugs for a specific organism or a group or organisms.

Some embodiments comprise conducting said process steps on a plurality of primary samples, taken from or derived from a plurality of individual organisms, and deriving at least in part from the thereby obtained data the information for a biomarker, which at least in part can quantify the actual efficacy of an administered drug or mix of drugs for a specific organism or a group or organisms.

Some embodiments comprise conducting said process steps on a plurality of primary samples, taken from or derived from a plurality of individual organisms, and deriving at least in part from the thereby obtained data the information for a biomarker, which at least in part can predict side effects of a drug for a specific organism or group of organisms.

Some embodiments comprise conducting said process steps on a single or a plurality of primary samples, taken from or derived from an individual organisms, and deriving at least in part from the thereby obtained data the occurring onset, or stage, or type of a disease in said individual organism.

Some embodiments comprise conducting said process steps on a single or a plurality of primary samples, taken from or derived from an individual organisms, and deriving at least in part from the thereby obtained data the expected efficacy of a drug or mix of drugs for said individual organism.

Some embodiments comprise conducting said process steps on a single or a plurality of primary samples, taken from or derived from an individual organisms, and deriving at least in part from the thereby obtained data the actually occurring efficacy of an administered drug or mix of drugs for said individual organism.

Some embodiments comprise conducting said process steps on a single or a plurality of primary samples, taken from or derived from an individual organisms, and deriving at least in part from the thereby obtained data at least some of the side effects of a specific drug or mix of drugs for said individual organism.

Some embodiments comprise conducting said process steps on a single or a plurality of primary samples, taken from or derived from an individual organisms,
and deriving at least in part from the thereby obtained data the information which quantifies at least in part the actual efficacy and/or at least some of the side effects of the administered drug or mix of drugs for a specific organism,
and with the times for sample processing short enough (in some embodiments within minutes or hours) to enable several such measurements during the time the drug or drug mix is administered (in some embodiments hours or days),
and in some embodiments furthermore changing the amount or composition of the administered drug or mix of drugs such that at least one desirable target is at least approximated (such as minimizing or reducing at least some of the side effects, enhancing efficacy, required duration of the treatment, etc., or any combination thereof).

Some embodiments comprise conducting said process steps on a plurality of primary samples, taken from, or consisting of, or derived from a single or a plurality of cells, and deriving at least in part from the thereby obtained data the information for a biomarker, which at least in part can indicate the occurring onset, or stage, or type of a disease.

Some embodiments comprise conducting said process steps on a plurality of primary samples, taken from, or consisting of, or derived from a single or plurality of cells, and deriving at least in part from the thereby obtained data the information for a biomarker, which at least in part can predict the expected efficacy of a drug for a specific organism or group of organisms.

Some embodiments comprise conducting said process steps on a plurality of primary samples, taken from, or consisting of, or derived from a single or a plurality of cells, and deriving at least in part from the thereby obtained data the information for a biomarker, which at least in part can quantify actual efficacy of an administered drug or mix of drugs for a specific organism or group of organisms.

Some embodiments comprise conducting said process steps on a plurality of primary samples, taken from, or consisting of, or derived from a single or a plurality of cells, and deriving at least in part from the thereby obtained data the information for a biomarker, which at least in part can predict side effects of a drug or mix of drugs for a specific organism or group of organisms.

Some embodiments comprise conducting said process steps on a single or a plurality of primary samples, taken from, or consisting of, or derived from a single or plurality of cells,
and deriving at least in part from the thereby obtained data the information which quantifies at least in part the actual efficacy and/or side effects of the administered drug or mix of drugs for a specific organism,
and with the times for sample processing short enough (in some embodiments within minutes or hours) to enable several such measurements during the time the drug or drug mix is administered (in some embodiments hours or days),
and in some embodiments changing the amount or composition of the drug or mix of drugs such that at least one desirable target is at least approximated (such as minimizing or reducing side effects, enhancing efficacy, required duration of the treatment, etc., or any combination thereof).

Some embodiments comprise conducting said process steps on a plurality of primary samples and using at least in part the thereby obtained data

for any one or any combination both of:
obtaining information constituting a biomarker
for any one or any combination both of:
   the onset, stage, or type of a disease in a cell or organism,
   the expected efficacy of a drug, or
   or the actual efficacy of an administered drug, or
obtaining information about the onset, or stage, or type a disease in a plurality of cells or plurality of organisms,
   and conducting these processes using a plurality of systems, which at least in part perform said first, second, third, and forth process steps,
   and which are predominantly housed within relative close spatial proximity.

Some embodiments further comprise using any of the obtained and/or derived information to develop and subsequently use at least one diagnostic test, which is at least in part is based on one or both of the detection and quantification of at least one affinity-based molecular binding event.

Some embodiments comprise conducting said process steps on a plurality of primary samples, taken from or derived from a single or a plurality of one or both of stem cells and cells derived therefrom, and deriving at least in part information about the differentiation of said stem cells into specific cell types, and using said information to derive diagnostic tests.

Some embodiments comprise conducting said process steps on a plurality of primary samples, taken from or derived from a single or a plurality of one or both of stem cells and cells derived therefrom, and deriving at least in part information about the differentiation of said stem cells into specific cell types, and using said information to guide, assist in, or derive the composition of drugs, or mixes of drugs, or other forms of treatment.

Some embodiments comprise conducting said process steps on a plurality of primary samples, taken from or derived from a single or a plurality of one or both of stem cells and cells derived therefrom, and deriving at least in part information about the differentiation of said stem cells into specific cell types, and using said information to guide, assist in, or derive the composition of drugs, or other chemical, or methods to induce and or direct cell differentiation.

Some embodiments comprise conducting said process steps on a plurality of primary samples, and using the obtained information to monitor or control the process of induced cell differentiation.

Some embodiments comprise conducting said process steps on a plurality of primary samples, taken from or derived from a single or a plurality of one or both of stem cells and cells derived therefrom, and deriving at least in part information about the differentiation of said stem cells into specific cell types, and using said information to guide, assist in, or derive the composition of drugs or other methods to correct undesirable effects or developments during cell differentiation.

Some embodiments comprise conducting said process steps on a plurality of primary samples, taken from or derived from a single or a plurality of one or both of stem cells and cells derived therefrom, and deriving at least in part information about the differentiation of said stem cells into specific cell types, and using said information to guide, assist in, or derive the composition drugs or other methods, which at least in part can reverse said process of differentiation.

Some embodiments comprise conducting said process steps on a plurality of primary samples, and using the obtained information to monitor or control the process of reversal of cell differentiation.

Some embodiments comprise using said derived methods or drugs to produce for a specific organism any one or any combination of:
predominantly cells of a predominantly specific type,
predominantly tissue of a predominantly specific type,
predominantly organs, and
   based on cellular samples previously taken from said organism.

The disclosed invention to analyze complex mixtures organic molecules, specifically of biopolymers, may in some embodiments be used to determine at least in part the nature of biological hazards, bioterrorism threats, or biological weapons, including those based on bacteria and viruses. In such cases samples taken from said hazards, objects associated with said threats, or substances used in said weapons are being subjected to the disclosed rigorous method.

Correspondingly, the disclosed invention to analyze complex mixtures organic molecules, specifically of biopolymers, may in some embodiments be used to select, develop, and or optimize countermeasures to biological hazards, bioterrorism threats, or biological weapons, including those based on bacteria and viruses.

The disclosed invention to analyze complex mixtures organic molecules, specifically of biopolymers, may in some embodiments be used to determine a biological marker, which serves to assess the expected performance level of a human or animal for a specific task or for a class or problems. One specific example may be to select a smaller group of humans or animals from a larger group of humans or animal, the smaller group being expected to have a higher probability of succeeding or otherwise having a higher level of performance in a subsequently conducted task or class of problems.

Furthermore, the disclosed invention to analyze complex mixtures organic molecules, specifically of biopolymers, may in some embodiments be used to guide the selection and or administering of drugs or mix of drugs to enhance the performance level of a human or animal for a specific task or for a class or problems. In some embodiments this may include increasing the probability of a human or animal to survive a certain task, class of tasks, or procedure, including but not limited to for example surgical operations or other medical treatments, or extreme environmental conditions, or other stress situations.

Therefore, it becomes clear that massive, high-throughput analysis of proteins is the next revolution in molecular biology and molecular diagnostics, which will happen after cheap DNA sequencing.

It may seem impractical for clinical application of protein biomarkers to ever materialize, if such considerable technical effort is required. But this concern is ill founded. First, one has to realize the incredible decline in the cost in DNA sequencing (from hundreds of million dollars to about $1000 for a human genome), which has been achieved during the last 15 years as a result of improvements in instrumentation. It is also possible, that some further cost reductions of the envisioned high-throughput, intrinsically quantitative protein analyzing systems will be achieved thanks to mass production of instruments.

But even if not, even if a machine, which can completely analyze and quantify on the order of 10⁵ proteins per day, would keep costing several million dollars to built, there is a large industry, which demonstrates that business is still viable, as long as the throughput is high enough.

Very few users of modern smart phones are aware of which kind of instrument and technology they ultimately owe the fact, that they can carry an amount of computing power in their pockets, that was only available to users of supercomputers 25 years ago: High-resolution, high-speed steppers, built by a handful of companies around the world, like Canon. These technical marvels, the size of a small room and costing up to $20M, are needed to imprint the electronic layout of integrated circuits onto the silicon wafer during several process steps. Ultimately, these are high-throughput information transfer machines. Several of these steppers are typically needed to set up a chip fab, which now typically costs between $1 and $2 billions. Yet thanks to the high throughput, individual chips can be fabricated at prices ranging from a few dollars to a hundred dollar (some CPUs and GPUs), and still be profitably sold.

Thus the same model could be applied to wide-spread analysis of complex protein mixtures. Specifically, under one scenario would such an approach be inevitable. It is not inconceivable that it eventually turns out that due to genetic reasons for some diseases general protein biomarkers, which are identical for all humans, do not exist. (For general markers cheap antibody-based test can subsequently be developed.) If for said conditions the corresponding protein biomarkers apply only to small, genetically very similar groups of individuals, one may ultimately come to the conclusion that in order to detect the onset of these conditions it is in fact best that every individual becomes their own standard in terms of what protein abundance is normal. In this scenario it would become custom, that perhaps every 6 to 12 months a comprehensive (serum) protein profile is acquired, starting at a very early age. One can imagine such a method like a molecular oscilloscope with 100,000 or 1 million channels, which must be able to sustain quantitative accuracy of probably better than 10% over decades. This can technically be accomplished if the disclosed robotic high-throughput systems for protein analysis are used on a large scale, comparable to the microelectronics industry today.

Currently, some acutely ill patients in hospitals have blood samples taken perhaps once a day and analyzed for a very few parameters. It would be of very high clinical value to take the same blood sample and subject it to such a comprehensive protein analysis. For example, the effectiveness and optimal dose of administered drugs could essentially be observed in real time.

Assuming the required effort for the above described system (which can completely analyze and quantify on the order of 10⁵ proteins per day) is $4M and it is written off after 5 years. It will produced 1825 protein profiles at a cost of about $2200 per profile (ignoring operational cost). This is clearly already within an acceptable range to monitor patients on a daily basis under extreme circumstances in a hospital or to be conducted in larger (e. g. annual) intervals for routine screening.

The mentioned search for a biomarker on a 2·100 cohort would thus correspond to about $440 k cost for instrument time, which is small compared to the potential value of a discovered bio marker.

After having developed relatively inexpensive high throughput DNA sequencing technology and with the ‘$1000 human genome’ achieved, the next technologically feasible long-term goal, likely achievable within less than a decade, must now be a “$1000 protein profile”. No single physical instrumentation project would now be more important for the understanding of the molecular biology of life and of human health. In some embodiments the disclosed invention provides elements to at least approach or even realize this and other goals.

Finally it shall be noted that by the citation of various references in this document the applicant does not admit that any particular reference is “prior art” to his invention.

Headings are included herein for reference and to aid in locating various sections. These headings are not intended to limit the scope of the described concepts.

Furthermore, while particular embodiments of the invention and variations thereof have been described in detail, other modifications and methods of using the disclosed invention will be apparent to those of skill in the art. Accordingly, it should be understood that various applications, modifications, and substitutions may be made of equivalents without departing from the spirit of the invention or the scope of the claims. Various terms have been used in the description to convey an understanding of the invention; it will be understood that the meaning of these various terms extends to common linguistic or grammatical variations or forms thereof. It will also be understood that when terminology referring, for example to physical equipment, hardware, or software may have used trade names or common names, that these names are provided as contemporary examples, and the invention is not limited by such literal scope. Terminology that is introduced at a later date that may be reasonably understood as a derivative of a contemporary term or designating of a subset of objects embraced by a contemporary term will be understood as having been described by the now contemporary terminology.

What is claimed:

1. A method of analyzing at least one sample comprising
releasing from said at least one sample at least some organic molecules or fragments thereof,
ionizing at least some of said organic molecules or fragments thereof at least in part by using multiply positively charged ions of relatively low atomic mass, produced by removal of more than one electron from atoms, and
subjecting at least one of the thereby produced multiply positively charged organic molecules or fragments thereof to at least one process step,
which at least in part achieves any one or any combination of:
separation of said multiply positively charged organic molecules or fragments thereof; or
determination of the molecular mass-to-charge ratios of said multiply positively charged organic molecules or fragments thereof; or
determination of the abundance of said multiply positively charged organic molecules or fragments thereof corresponding to molecular specific mass-to-charge ratios.

2. Method according to claim 1, further comprising
using said determination of said mass-to-charge ratios of said multiply positively charged organic molecules or fragments thereof
to at least in part determine any single or any combination of:
an amino or nucleic acid sequence; or
any chemical modification on any such amino or nucleic acid; or
any other molecule, weakly bound to any such amino or nucleic acid.

3. Method according to claim 2 further comprising
deriving at least in part from the thereby obtained data
the information for a biomarker,
which at least in part can indicate the onset, or stage, or type of a disease.

4. Method according to claim 2 further comprising
deriving at least in part from the thereby obtained data
the information for a biomarker,
which at least in part can one or both of
predict the expected efficacy of a drug, or
determine the actual efficacy of a drug
for a specific organism or group of organisms.

5. Method according to claim 2 further comprising
deriving at least in part from the thereby obtained data
the information for a biomarker,
which at least in part can predict side effects of a drug for a specific organism or group of organisms.

6. Method according to claim 2 further comprising
deriving at least in part from the thereby obtained data
the information to assess the expected performance level
of a human or animal for a specific task or for a class or problems.

7. Method according to claim 2 further comprising
deriving at least in part from the thereby obtained data
information about the differentiation of stem cells into specific cell types,
and using said information to derive
at least one drug or at least one method to induce cell differentiation.

8. Method according to claim 2 further comprising
deriving at least in part from the thereby obtained data
information to monitor or control the process of induced cell differentiation.

9. Method according to claim 2 further comprising
deriving at least in part from the thereby obtained data
the nature of a biological hazard, bioterrorism threat, or a biological weapon,
including those based on bacteria and viruses.

10. Method according to claim 2 further comprising
deriving at least in part from the thereby obtained data
countermeasures to a biological hazard, a bioterrorism threat, or a biological weapon, including those based on bacteria and viruses.

11. A method of analyzing at least one sample comprising
releasing from said at least one sample at least some organic molecules or fragments thereof,
ionizing at least some of said organic molecules or fragments thereof at least in part by using bare atomic nuclei, i.e., multiply positively charged ions of relatively low atomic weight, produced by removal of all electron from atoms, and
subjecting at least one of the thereby produced multiply positively charged organic molecules or fragments thereof to at least one process step,
which at least in part achieves any one or any combination of:
separation of said multiply positively charged organic molecules or fragments thereof; or
determination of the molecular mass-to-charge ratios of said multiply positively charged organic molecules or fragments thereof; or
determination of the abundance of said multiply positively charged organic molecules or fragments thereof corresponding to specific molecular mass-to-charge ratios.

12. Method according to claim 11, further comprising
using said determination of said mass-to-charge ratios of said multiply positively charged organic molecules or fragments thereof
to at least in part determine any single or any combination of:
an amino or nucleic acid sequence; or
any chemical modification on any such amino or nucleic acid; or
any other molecule, weakly bound to any such amino or nucleic acid.

13. Method according to claim 12 further comprising
deriving at least in part from the thereby obtained data
the information for a biomarker,
which at least in part can indicate the onset, or stage, or type of a disease.

14. Method according to claim 12 further comprising
deriving at least in part from the thereby obtained data
the information for a biomarker,
which at least in part can one or both of
predict the expected efficacy of a drug, or
determine the actual efficacy of a drug
for a specific organism or group of organisms.

15. Method according to claim 12 further comprising
deriving at least in part from the thereby obtained data
the information for a biomarker,
which at least in part can predict side effects of a drug for a specific organism or group of organisms.

16. Method according to claim 12 further comprising
deriving at least in part from the thereby obtained data
the information to assess the expected performance level of a human or animal for a specific task or for a class or problems.

17. Method according to claim 12 further comprising
deriving at least in part from the thereby obtained data
information about the differentiation of stem cells into specific cell types,
and using said information to derive
at least one drug or at least one method to induce cell differentiation.

18. Method according to claim 12 further comprising
deriving at least in part from the thereby obtained data
information to monitor or control the process of induced cell differentiation.

19. Method according to claim 12 further comprising
deriving at least in part from the thereby obtained data
the nature of a biological hazard, bioterrorism threat, or a biological weapon, including those based on bacteria and viruses.

20. Method according to claim 12 further comprising
deriving at least in part from the thereby obtained data
countermeasures to a biological hazard, a bioterrorism threat, or a biological weapon, including those based on bacteria and viruses.
